(19) United States
(12) Patent Application Publication
(10) Pub. No.: US 2012/0310051 A1
(43) Pub. Date: Dec. 6, 2012

(54) Systems And Methods For Signal Rephasing Using The Wavelet Transform

(75) Inventors: Paul Addison, Edinburgh (GB); Scott McGonigle, Haddington (GB); James Watson, Dunfermline (GB)

(73) Assignee: Nellcor Puritan Bennett Ireland, Mervue (IE)

(21) Appl. No.: 13/149,755

(22) Filed: May 31, 2011

(51) Int. Cl. A61B 5/08; A61B 5/1455; A61B 5/00

(52) U.S. Cl. 600/301; 600/300; 600/323

(57) ABSTRACT

Methods and systems are disclosed for defining a physiological parameter. A first physiological signal is transformed into in a complex transform space, the transformed signal having a magnitude and a phase. The transformed signal is rotated by altering its phase. The rotated signal is inverted, and the inverted signal is aligned in phase with a second physiological signal. The aligned inverted signal and the second physiological signal are combined to form a combined signal indicative of the physiological parameter.

### SUMMARY OF THE DISCLOSURE

The present disclosure relates to signal processing and analysis and, more particularly, the present disclosure relates to systems and methods for rephasing a signal using a complex transform.

In certain aspects, the systems and methods provide signal processing steps to determine useful information from a measured signal. In some applications, these signal processing steps include rephasing a signal or a component of a signal to align it with another signal or signal component. In connection with deriving useful information (e.g., clinical information) from a physiological signal, signals may be aligned with each other in phase so that a composite signal and/or physiological parameters can be determined. Rather than simply shifting one signal with respect to another signal by, for example, introducing a time delay into one signal, the phase shift can be carried out by transforming one signal in a complex transform space, rotating the transformed signal by the phase difference between the first signal and the other signal, and performing an inverse transform of the rotated transformed signal. This process has the added benefit of reducing noise effects in the method compared to, e.g., time delay methods. This process of rephasing and aligning signals, as described in more detail below, may be used to align respiratory components of a physiological signal to derive a composite respiration signal and determine a respiration rate.

According to one aspect, the disclosure relates to methods for determining a physiological parameter from at least two physiological signals which are not aligned in phase. In order to determine the physiological parameter, the physiological signals are first aligned in phase. There are several reasons why physiological signals may not be aligned in phase. For example, a single received physiological signal may contain multiple component signals which are not aligned in phase, or multiple physiological signals may have been received from different parts of a subject's body or at different times. Using the systems and methods described herein, multiple physiological signals or components of physiological signals can be aligned in phase to provide information indicative of physiological parameters, such as a respiration signal.

To derive a physiological parameter from unaligned signals, a first physiological signal is transformed into in a complex transform space. The transformed signal has a magnitude and a phase. The signal may be transformed using, for example, a continuous wavelet transform or a short time Fourier transform. Transforming the signal into the complex transform space may reduce the noise of the signal. The transformed signal is then rotated by altering its phase. In certain embodiments, the phase shift value used for altering the phase of the transformed signal is a minimum value of a median of the distances between corresponding peaks of the first and second physiological signals. Finally, the rotated signal is inverted; the inverted signal is aligned in phase with a second physiological signal. The aligned inverted signal and the second physiological signal can be combined to form a combined or composite signal, which is indicative of the physiological parameter. In certain embodiments, signals are combined by normalizing and summing the signals.

In certain embodiments, at least one of the physiological signals is a photoplethysmograph (PPG) signal measured by a pulse oximeter. The PPG signal may include at least one respiratory component. For example, the PPG signal may include at least one of a baseline modulation signal, an amplitude modulation signal, and respiratory sinus arrhythmia modulation signal. The physiological parameter may be a parameter indicative of respiration, such as a respiration rate.

In certain embodiments, the method further comprises aligning at least a third physiological signal to the second physiological signal. To do this, a process similar to the process performed on the first physiological signal is performed: the third signal is transformed into a complex transform space, this transformed signal is rotated by altering its phase, and this rotated signal is inverted. A phase shift value by which the third signal is altered may be different from the phase shift value by which the first signal is altered.

In another aspect, the disclosure relates to systems for displaying a physiological parameter. Such systems include a signal input for receiving at least one physiological signal of a subject from a sensing device, such as a pulse oximeter. Electronic processing equipment is coupled to the signal input for receiving the at least one physiological signal, aligning a physiological parameter with a reference physiological parameter, and generating a combined signal indicative of the physiological parameter. The system also includes a monitor for displaying a physiological parameter representative of the combined signal. The monitor may also display the combined signal. The system may be configured to perform any of the methods described above.

In another aspect, the disclosure relates to a computer-readable medium having computer program instructions recorded thereon for carrying out methods for defining a physiological parameter including the methods described above.

Several methods and systems for aligning physiological signals for defining a physiological parameter are disclosed herein. In a patient monitoring setting, the physiological parameter determined may be used in a variety of clinical applications, including within diagnostic and predictive models, and may be recorded and/or displayed by a patient monitor.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIG. 3 is an illustrative plot showing respiratory signal components derived from a PPG signal in accordance with an embodiment;

FIGS. 4a through 4c illustrate aspects of a signal alignment by transformation and rotation;

FIG. 5 is a flow chart of illustrative steps for aligning physiological signals in phase and combining the signals in accordance with an embodiment;

FIGS. 6a and 6b are flow charts of illustrative steps involved in performing an inverse continuous wavelet transform in accordance with embodiments;

FIG. 7 is a block diagram of an illustrative continuous wavelet processing system in accordance with some embodiments;

FIG. 8 is an illustrative plot showing the respiratory signal components from FIG. 3 aligned in phase in accordance with an embodiment;

FIG. 9 is an illustrative plot showing a combined signal laid over the aligned respiratory signal components from FIG. 8 in accordance with an embodiment;

FIG. 10a is a plot of a PPG signal;

FIG. 10b is illustrates a signal having undergone a phase shift; and

FIG. 10c illustrates a signal where low-frequency components have undergone a phase shift, but high-frequency components have not.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may transmit signals from a light sensor that is placed at a site on a patient, typically a fingertip, toe, forehead or earlobe, or in the case of a neonate, across a foot. The oximeter may pass light using the light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
I_{O}=intensity of light transmitted;
s=oxygen saturation;
β_{O}, βᵣ empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and red

log I=log I_{O}−(sβ_{O}+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{o} ( {λ}_{IR} ) - {β}_{r} ( {λ}_{IR} ) ) - \frac{d log I ( {λ}_{IR} )}{d t} ( {β}_{o} ( {λ}_{R} ) - {β}_{r} ( {λ}_{R} ) )}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{O} ( {λ}_{IR} ) - {β}_{r} ( {λ}_{IR} ) ) - {β}_{O} ( {λ}_{R} ) + {β}_{R} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{\frac{d I}{d t}}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)[I(t₂,λ_{IR})−I(t₁/λ_{IR})]I(t₁,λ_{R})

y(t)=[I(t₂,λ_{R})−I(t₁/λ_{R})]I(t₁,λ_{IR})

y(t)=Rx(t) (8)

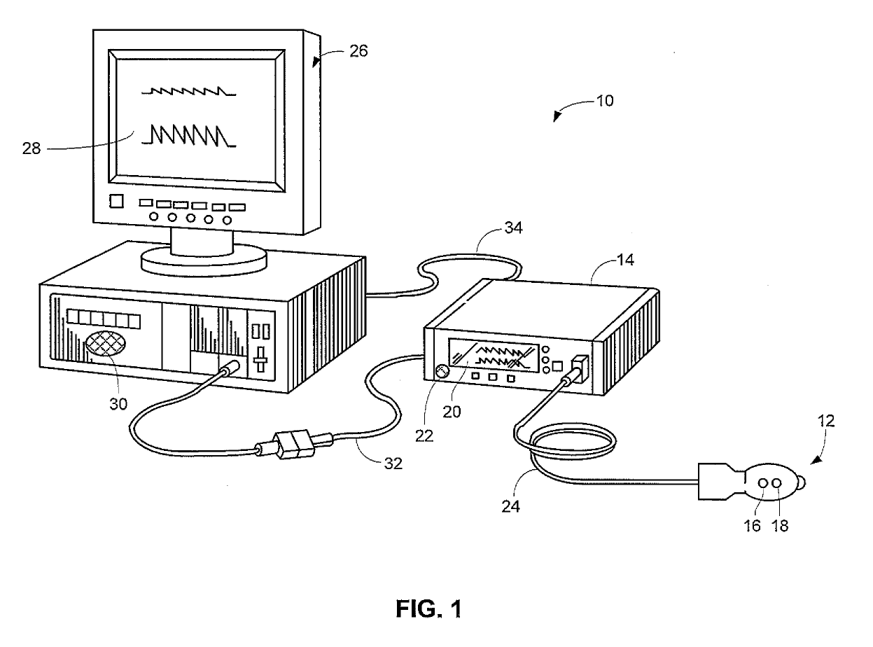

FIG. 1 is a perspective view of an embodiment of a pulse oximetry system 10. System 10 includes a sensor 12 and a pulse oximetry monitor 14. Sensor 12 includes an emitter 16 for emitting light at two or more wavelengths into a patient's tissue. A detector 18 is provided in sensor 12 for detecting the light originally from emitter 16 that emanates from the patient's tissue after passing through the tissue.

According to another embodiment, and as will be described, the system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to certain embodiments, emitter 16 and detector 18 are on opposite sides of a digit such as a finger or toe, in which case the light that is emanating from the tissue has passed completely through the digit. The emitter 16 and detector 18 may also be arranged so that light from emitter 16 penetrates the tissue and is reflected by the tissue into detector 18, such as a sensor designed to obtain pulse oximetry data from a patient's forehead.

In certain embodiments, the sensor or sensor array are connected to and draw power from monitor 14. The sensor may also be wirelessly connected to monitor 14 and include its own battery or similar power supply (not shown). Monitor 14 may be configured to calculate physiological parameters based at least in part on data received from sensor 12 relating to light emission and detection. In alternative embodiments, the calculations may be performed on the monitoring device itself, and the result of the oximetry reading may be passed to monitor 14. Further, monitor 14 may include a display 20 configured to display the physiological parameters or other information about the system. In the embodiment shown, monitor 14 also includes a speaker 22 to provide an audible sound that may be used in various other embodiments, such as for example, sounding an audible alarm in the event that a patient's physiological parameters are not within a predefined normal range.

In certain embodiments, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 also includes a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by pulse oximetry monitor 14 (referred to as an “SpO₂” measurement), pulse rate information from monitor 14, respiration information generated by monitor 14, and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

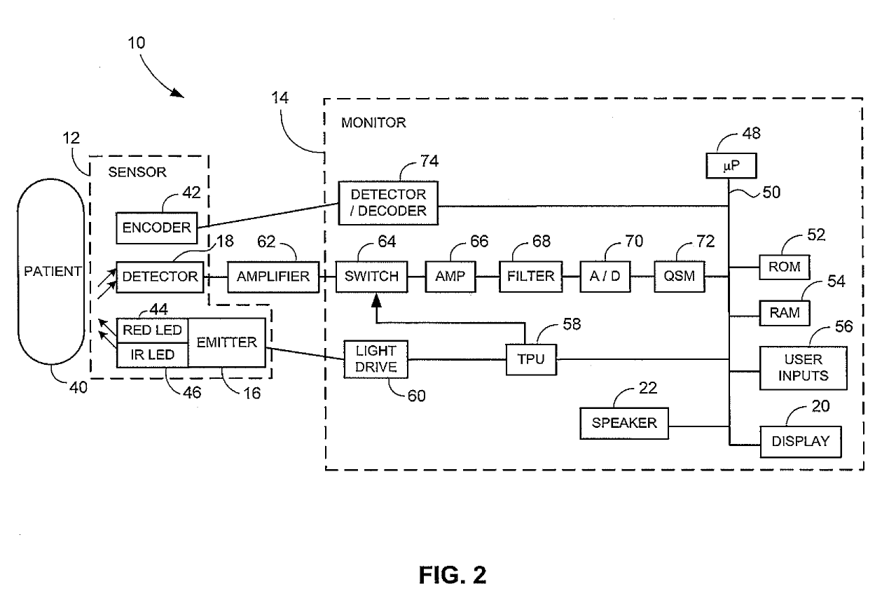

FIG. 2 is a block diagram of a pulse oximetry system, such as pulse oximetry system 10 of FIG. 1, which is coupled to a patient 40. Certain illustrative components of sensor 12 and monitor 14 are illustrated in FIG. 2. Sensor 12 includes emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In certain embodiments, the RED wavelength is between about 600 nm and about 700 nm, and the IR wavelength is between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” refers to energy produced by radiative sources and may include one or more of ultrasound, radio, microwave, millimeter wave, infrared, visible, ultraviolet, gamma ray or X-ray electromagnetic radiation. As used herein, light may include any wavelength within the radio, microwave, infrared, visible, ultraviolet, or X-ray spectra. Any suitable wavelength of electromagnetic radiation may be appropriate for use with the present techniques. Detector 18 may be chosen to be specifically sensitive to the chosen targeted energy spectrum of the emitter 16.

In certain embodiments, detector 18 is configured to detect the intensity of light at the RED and JR wavelengths. Alternatively, each sensor in the array may be configured to detect an intensity of a single wavelength. In operation, light enters the detector 18 after passing through the patient's tissue 40. The detector 18 converts the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 sends the signal to monitor 14, where physiological parameters are calculated based on the absorption of the RED and IR wavelengths in the patient's tissue 40.

In certain embodiments, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In certain embodiments, signals from detector 18 and encoder 42 are transmitted to monitor 14. In the embodiment shown, monitor 14 includes a general-purpose microprocessor 48 connected to an internal bus 50. Microprocessor 48 is adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 provides timing control signals to light drive circuitry 60, which controls when emitter 16 is illuminated, and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In certain embodiments, there may be multiple separate parallel paths having amplifier 66, filter 68, and A/D converter 70 for multiple light wavelengths or spectra received.

In certain embodiments, microprocessor 48 determines the patient's physiological parameters, such as SpO₂, pulse rate, and respiration, using various algorithms and/or look-up tables based on the value of the received signals and/or data corresponding to the light received by detector 18. The microprocessor 48 may be used for signal processing. For example, microprocessor 48 may align two physiological signals, generate a combined signal, and generate a physiological parameter from the combined signal. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In certain embodiments, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic radiation from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other biosignals including, but not limited to, electrocardiogram, electroencephalogram, electrogastrogram, electromyogram, heart rate signals, pathological sounds, ultrasound, or any other suitable biosignal, and/or any combination thereof.

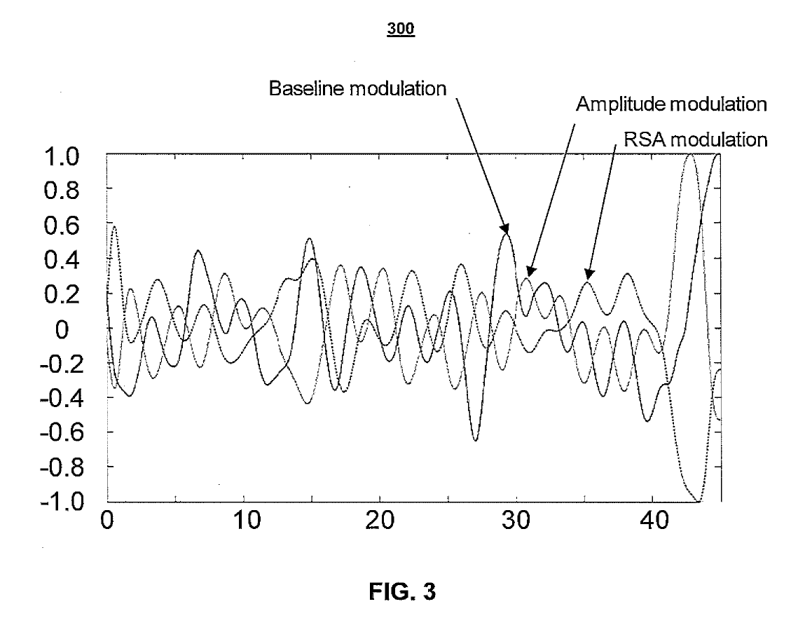

In certain embodiments, the present disclosure is applied to respiration signals. A PPG signal includes, among other components, a cardiac component and multiple lower-frequency respiration modulations. These respiration modulations include a baseline modulation signal, an amplitude modulation signal, and a respiratory sinus arrhythmia (RSA) signal. An illustration of these signals decoupled from the cardiac signal is shown in FIG. 3. The horizontal axis represents time, and the vertical amplitude represents the amplitude of the signal. As shown in FIG. 3, these three signals are out of phase, meaning that at any given point in time, each signal's phase is different from the phases of the other signals. In order to determine an overall respiration signal, parameters characteristic of a patient's respiration, and other information indicative of a patient's respiration, the three signals may be aligned in phase.

The baseline modulation signal represents a change in intrathoracic pressure. Change in intrathoracic pressure affects the amount of venous blood drawn to the heart and causes changes in the amount of venous blood flow. The amplitude modulation signal correlates to the volume of blood being pumped by arterial pulses. A person's breathing affects the volume of blood that is pumped. RSA is variation in the heart rate that occurs during a breathing cycle. When a person inhales, the heart rate increases; when a person exhales, the heart rate decreases. Since these variations affect different aspects of the circulatory system (venous flow, arterial flow, and pulse rate), they may not be aligned in phase, as seen in FIG. 3. However, to derive a single characteristic respiration signal or respiration parameter, it is often more accurate to use a combination of these signals rather than the individual signals. To this end, the respiration signals can be aligned in phase and then combined to form a composite respiration signal.

One method for aligning the signals is shifting the signals in time with respect to each other, i.e., adding a time delay to some of the signals. However, if the signals are time-shifted with respect to each other so that, for example, the peaks line up or the troughs line up, then noise present at a specific time in the signal may also shift and spread out over time. For example, if a patient wearing a pulse oximeter moves, it may create noise in all components of the PPG signal localized in time at the point in time that the patient moved. If one component of the PPG signal is time shifted with respect to another component, the noise will also shift. Then, if two components are combined to generate a combined signal, the noise would be present at two locations in the combined signal. So, time shifting signals before combining them may spread out noise over time. Therefore, a method which shifts the signal phase without moving the signal content may be preferable in some implementations.

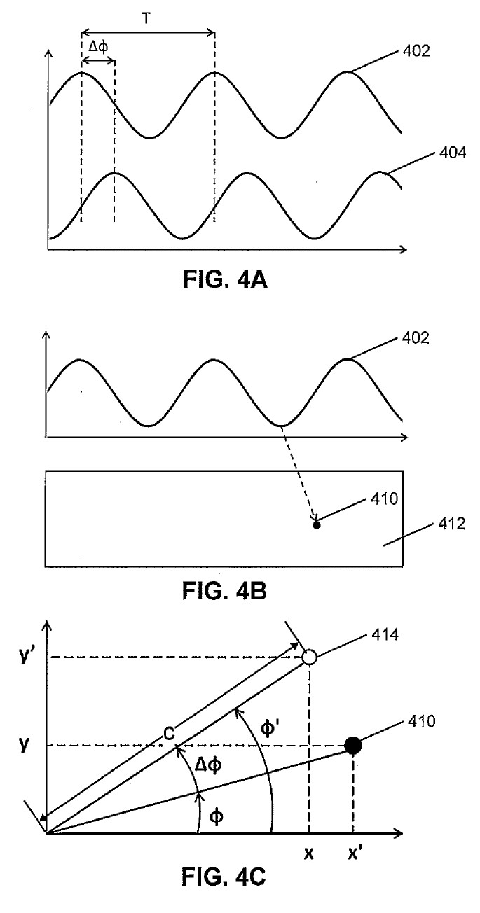

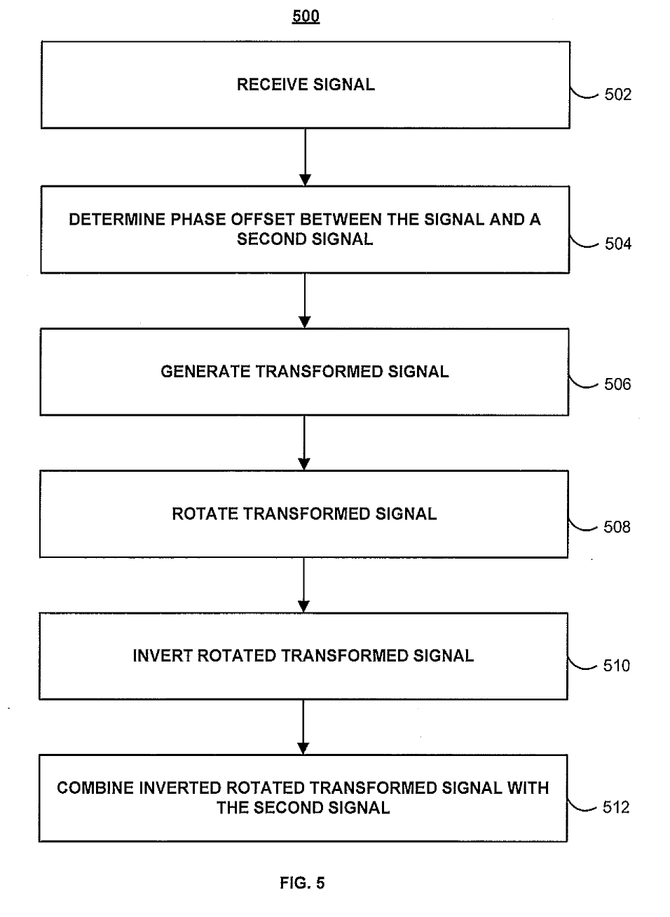

FIG. 4a through 4c illustrate schematically a method for aligning signals by determining a phase offset between the signals (FIG. 4a), transforming a point to wavelet space (FIG. 4b), and rotating a point in wavelet space (FIG. 4c). In particular, FIG. 4a shows a schematic of two signals 402 and 404, where one of the signals 402 is out of phase from the other signal 404 by a phase difference Δ□. The phase difference Δ□ is determined, for example, by one of the methods for finding Δ□ are described in relation to step 504 of FIG. 5. The signal 402 also has a period T which is similar to or the same as the period of signal 404. FIG. 4b shows performing a transform, such as a continuous wavelet transform, of a point on the signal 402 into a complex transform space. Each point of the signal 402 is transformed into a point, such as point 410, on the transform surface 412. Each point on the transform surface is a complex number c=x+iy that can be described by a magnitude |c| let and phase □.

The point 410 on the transform surface 412 is shown on x-y axes in FIG. 4c. The point 410 is rotated by the phase difference Δ□ to new point 414. Mathematical techniques for rotating the signal are discussed in relation to step 508 of FIG. 5. As shown in FIG. 4c, the magnitude c is the same for both points 410 and 414; the phase changes to □′=□+Δ□. The method may be applied to each point on the complex transform space, such that this rotation operation is performed on each point. Once the phases have been shifted, the inverse transform is carried out on the rephased transform to produce the rephased signal. By shifting the phase of a signal this way, noise present in that signal can stay localized in time.

A flowchart for a method 500 of aligning and combining physiological signals, such as the aforementioned respiration signals, or other periodic signals is shown in FIG. 5. The method 500 includes the steps of receiving a first signal 502, determining the phase offset with respect to a second signal 504, transforming the first signal to generate a transformed signal 506, rotating the phase of the transformed signal 508, inverting the rotated transformed signal 510, and combining the inverted signal with the second signal 512. The steps of flowchart 500 may be performed by processor 48, or any other suitable processing device communicatively coupled to monitor 14. For example, the steps of flow chart 500 may be performed by a digital processing device, or implemented in analog hardware. It will be noted that the steps of flow chart 500 may be performed in any suitable order, and certain steps may be omitted entirely.

The steps of flowchart 500 may be executed over a sliding window of samples or time of a signal. For example, the steps of flowchart 500 may involve analyzing N samples of a signal, or the signal received over T units of time. The length of the sliding window over which the steps of flowchart 500 is executed may be fixed or dynamic. In certain embodiments, the length of the sliding window is based at least in part on the noise content of a signal. For example, the length of the sliding window may increase with increasing noise, as may be determined by a noise assessment. In certain embodiments, the steps of flowchart 500 are executed on one or more signals retrieved from memory. For example, the steps of flowchart 500 may involve analyzing one or more signals that were previously stored in ROM 52, RAM 52, and/or QSM 72 (FIG. 2(b)) in the past and are accessed by microprocessor 48 within monitor 14 to be processed.

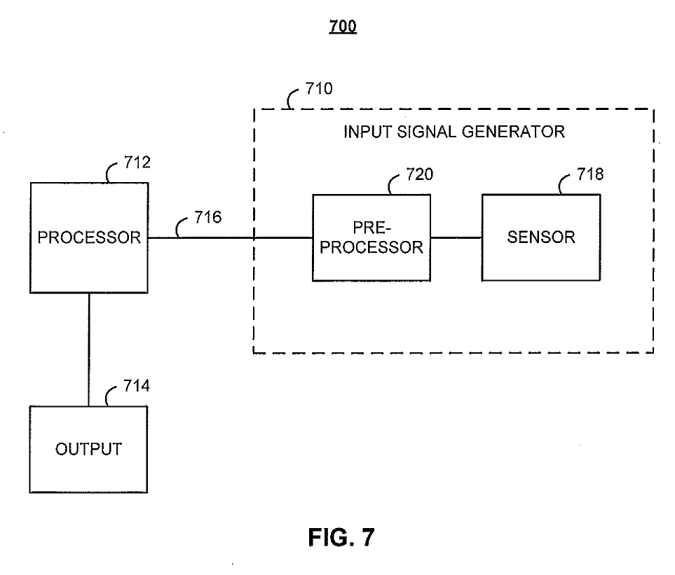

In step 502, the monitor 14 receives a signal (e.g., a PPG signal) from a source (e.g., patient 40). A received signal may be generated by sensor unit 12, which may itself include any of the number of physiological sensors described herein. The physiological signal may contain at least two component signals, such as a cardiac signal, a baseline modulation signal, an amplitude modulation signal, and an RSA signal, described in relation to FIG. 3. Pre-processing may be performed on the signal before or after it is received by the monitor at the sensor 12 or the monitor 14. Pre-processing techniques include one or more of filtering, transforming, compressing, and sampling. Pre-processing techniques and further examples of types of received signals are described in detail in relation to FIG. 7.

In step 504, the processor 48 of the monitor 14 determines a phase offset between one signal and another signal. One signal may be selected to be the reference signal having a reference phase (e.g., φ=0), and the phase offsets of the other signals can be determined with respect to the reference phase of the reference signal. If there are more than two signals, the phase offset between each of the non-reference signals and the reference signal may be different. The phase offset between each non-reference signal and the reference signal can be determined through any known method, such as cross-correlation of the two signals or an analysis of the relative positions of fiducial points on the two signals (e.g., peaks, troughs, etc.).

In certain embodiments, the phase offset between two signals is determined as follows. A time offset of the two signals is determined by finding the median of the distances in time between corresponding peaks of the signals. Also, the period of each of the signals can be determined by finding the median interpeak value of the signal. The two signals being aligned preferably have substantially the same period; if they do not exactly match, one of the periods can be used, or the two periods of the signals can be averaged. The phase shift is then found by dividing the median time offset between the two signals by the median period. The resulting value can be converted to degrees or radians by multiplying by 360° or 2π radians, respectively. For example, if the time delay between two signals is 2 seconds, and the phase of each signal is 8 seconds, then the phase shift in radians is found as follows:

$$Δ φ = \frac{2 seconds}{8 seconds} * 2 π radians = \frac{π}{2} radians$$

In degrees, the phase shift is 90°. This method for determining phase offset is less influenced by the range of frequencies in the signals than cross-correlation.

In step 506, the processor 48 of the monitor 14 generates a transformed signal of each non-reference signal by performing a transform of the signal into a complex transform space. In certain embodiments, a continuous wavelet transform is preferred because it is a highly redundant, high resolution transform; the continuous wavelet transform will be described in further detail below. Alternatively, any other suitable transform that would transform the signal into a complex space, such as a continuous-time short tome Fourier transform (SIFT), could be used. In certain embodiments, processor 48 may transform the signal into any suitable domain, for example, a Fourier, wavelet, spectral, scale, time, time-spectral, time-scale domain, or any transform space. In some embodiments, step 504 is performed after step 506, and the phase difference is determined in the transform space rather than in the time domain.

In step 508, the processor 48 of the monitor 14 generates a rotated transformed signal by rotating the transformed signal by the phase offset determined in step 504. This is shown schematically in FIG. 4c. Each point on the transform surface is a complex number with a magnitude |c| and a phase φ. In exponential form, the rotation of a complex number |c|e^{i(φ)} is carried out by altering the exponent: |c|e^{i(φ-Δφ)}. The rotation can also be performed using a rotation matrix according to the following formula, where x and y are defined according to c=x+iy and x′ and y′ are the rotated x and y components:

$$[ \begin{matrix}{x}^{′} \\ {y}^{′}\end{matrix} ] = [ \begin{matrix}cos Δ φ & - sin Δ φ \\ sin Δ φ & cos Δ φ\end{matrix} ] [ \begin{matrix}x \\ y\end{matrix} ] \qquad ( 9 )$$

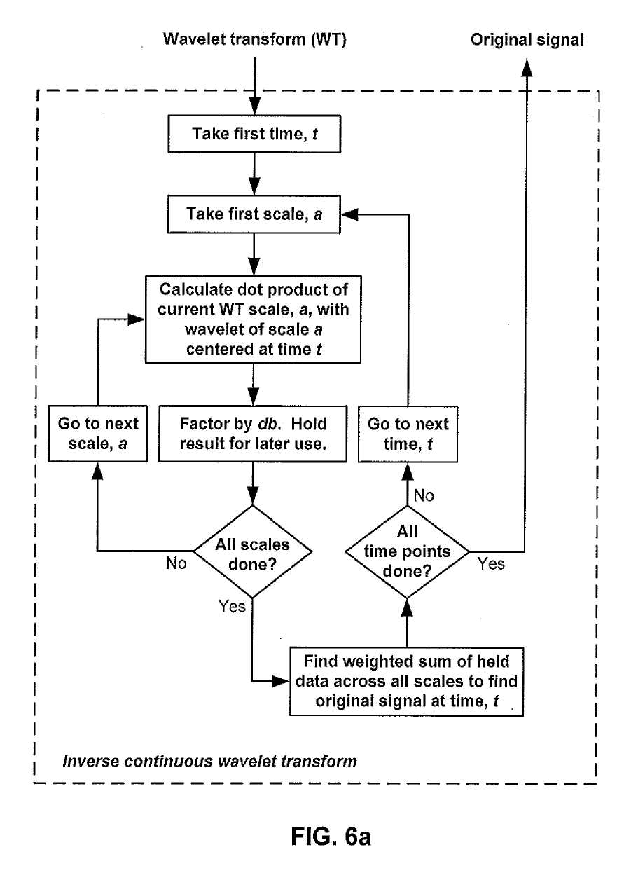

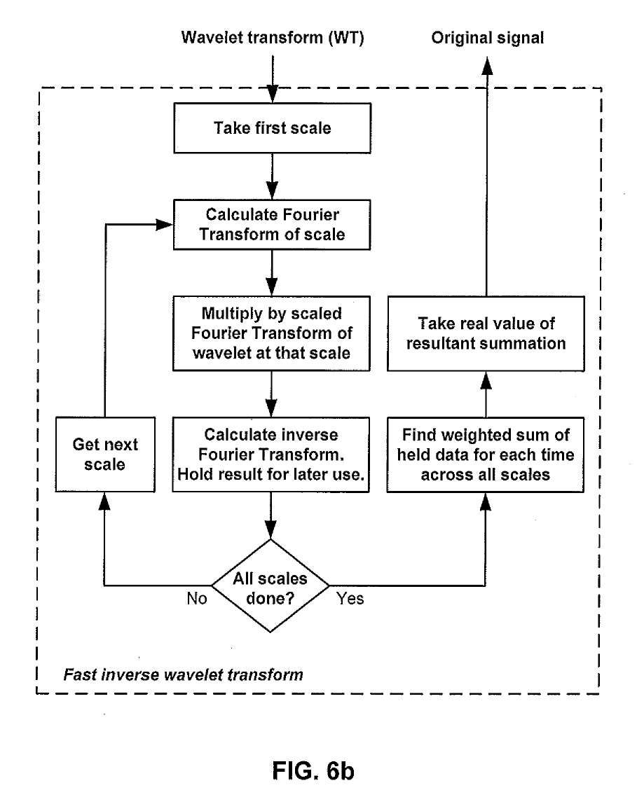

Once each point on the transform surface has been rotated, the rotated transformed signal is inverted in step 510. The inverse of the transform performed in step 506 is performed. This results in a rephased amplitude vs. time signal. Methods for inverting the continuous wavelet transform are described below in relation to FIGS. 6a and 6b. Steps 506 through 510 can be repeated for each additional non-reference signal of interest.

In step 512, the one or more inverted rotated transformed signals are combined with the reference signal to produce a combined, composite, or characteristic signal. The signals can be simply added; normalized and summed; normalized, weighted, and summed; or combined in any other suitable manner to produce the combined signal. Furthermore, a parameter indicative of the combined signal can be derived from the combined signal or any of the component signals. For example, the respiration rate can be found from a combined respiration signal by determining the frequency of that combined signal. Additionally or alternatively, respiration amplitude or volume can be calculated through analysis of the respiration signal. Any of the raw signals, rephased signals, combined signals, and physiological parameters can be displayed on the display 20 of the monitor 14 or display 28 of the monitor 26, sent to another monitoring device, and/or saved in memory.

A continuous wavelet transform of a PPG or other signal x(t) in accordance with the present disclosure may be defined as follows:

$$T ( a , b ) = \frac{1}{\sqrt{a}} {\int }_{- ∞}^{+ ∞} x ( t ) {ψ}^{*} ( \frac{t - b}{a} ) d t \qquad ( 10 )$$

where Ψ*(t) is the complex conjugate of the wavelet function Ψ(t), a is the dilation parameter of the wavelet and b is the location parameter of the wavelet. The transform given by equation (10) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. Various underlying mathematical details for implementation within a time-scale framework can be found, for example, in Paul S. Addison, The Illustrated Wavelet Transform Handbook (Taylor & Francis Group 2002), which is hereby incorporated by reference.

The continuous wavelet transform decomposes a signal using wavelets, which are generally highly localized in time. The continuous wavelet transform may provide a significantly higher resolution relative to discrete transforms. Thus, the continuous wavelet transform provides the ability to obtain more information from signals than the discrete wavelet transforms (or other discrete transforms). In addition, the continuous wavelet transform may provide more information from signals than typical frequency-only transforms such as Fourier transforms (or any other spectral techniques). Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with, dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as follows:

S(a,b)=|T(a,b)|² (11)

where ‘∥’ is the modulus operator. The scalogram may be resealed for useful purposes. One common resealing is defined as follows:

$${S}_{R} ( a , b ) = \frac{{\vert T ( a , b ) \vert}^{2}}{a} \qquad ( 12 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane is labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

The “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by:

$$f = \frac{{f}_{c}}{a} \qquad ( 13 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as follows:

Ψ=π^{-1/4}(e^{i2πf}^{₀}^{t}−e^{-(2πf}^{₀}⁾^{²}^{/2})e^{-t}^{²}^{/2} (14)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as follows:

$$ψ ( t ) = \frac{1}{{π}^{1 / 4}} {e}^{i 2 π {f}_{0} t} {e}^{- {t}^{2} / 2} \qquad ( 15 )$$

This wavelet is a complex wave within a scaled Gaussian envelope. While both definitions of the Morlet wavelet are included herein, the function of equation (15) is not strictly a wavelet as it has a non-zero mean (i.e., the zero frequency term of its corresponding energy spectrum is non-zero). However, it will be recognized by those skilled in the art that equation (15) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be used advantageously for aligning multiple signals. For example, PPG signals aligned in wavelet space may be used to provide clinically useful information within a medical device.

As discussed above, pertinent repeating features in the signal give rise to a time-scale band in wavelet space or a resealed wavelet space. For a periodic signal, such as pulse or respiration, this band remains at a constant scale in the time-scale plane. For many real signals, especially biological signals, the band may be non-stationary; varying in scale, amplitude, or both over time.

After the signal has been transformed and rotated, an inverse continuous wavelet transform is performed by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} Ψ ( \frac{t - b}{a} ) \frac{d a d b}{{a}^{2}} \qquad ( 16 )$$

This may also be written as follows:

$$x ( t ) = \frac{1}{{C}_{g}} {\int }_{- ∞}^{∞} {\int }_{0}^{∞} T ( a , b ) {Ψ}_{a , b} ( t ) \frac{d a d b}{{a}^{2}} \qquad ( 17 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 18 )$$

FIG. 6a is a flow chart of illustrative steps that may be taken to perform an inverse continuous wavelet transform in accordance with the above discussion. An approximation to the inverse transform may be made by considering equation (16) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 6b is a flow chart of illustrative steps that may be taken to perform an approximation of an inverse continuous wavelet transform. It will be understood that any other suitable technique for performing an inverse continuous wavelet transform may be used in accordance with the present disclosure.

In certain embodiments, respiration signals may be obtained through multiple sources, such as multiple pulse oximeters, which may be placed on different parts of the patient's body. Respiration signals may also have been collected or received at different times. If respiration signals are received from two different sensors, from two different parts of the body, or from different spans or time, the respiration signals will likely not be aligned in phase. The method for aligning signals described herein can be used to align in phase signals from signals from multiple sources or times, possibly in addition to aligning component signals from a single source.

FIG. 7 is an illustrative processing system. In this embodiment, input signal generator 710 generates an input signal 716. As illustrated, input signal generator 710 may include a pre-processor 720 coupled to sensor 718, which may provide as input signal 716, a PPG signal. It will be understood that input signal generator 710 may include any suitable signal source, signal generating data, signal generating equipment, or any combination thereof to produce signal 716. Signal 716 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

The signal 716 received by the processor 712 may be generated by a pre-processor 720 coupled between processor 712 and sensing device 718. A signal 716 may include multiple signals (e.g., first and second signals), or multiple signal components. Additionally, a signal received at step 502 may be a derived signal generated internally to processor 712. Accordingly, a received signal may be based at least in part on a filtered version of a signal 716, or a combination of multiple signals. For example, a received signal may be a ratio of two signals. A received signal may be a transformation of a signal 716, such as a continuous wavelet transformation of a signal 716. A received signal may be based at least in part on past values of a signal, such as signal 716, which may be retrieved by processor 712 from a memory such as a buffer memory or RAM 54. A received signal may include one or more signals within the received signal.

The signal received at step 502 (FIG. 5) is a PPG signal obtained from sensor 12 that is coupled to patient 40. A PPG signal may be obtained from input signal generator 710, which may include pre-processor 720 coupled to sensor 718, which may provide as input signal 716 a PPG signal. In certain embodiments, a PPG signal may be obtained from patient 40 using sensor 12 or input signal generator 710 in real time. In certain embodiments, a PPG signal may have been stored in ROM 52, RAM 52, and/or QSM 72 (FIG. 2) in the past and may be accessed by microprocessor 48 within monitor 14 to be processed. One or more PPG signals may be received as input signal 716 and may include one or more of a Red PPG signal and an IR PPG signal. In certain embodiments, a first signal may be a Red PPG signal, and a second signal may be an IR PPG signal. In certain embodiments, a first and second signal may be different types of signals (e.g., a blood pressure signal and a pulse rate signal). In certain embodiments, a first and second signal may be obtained by first and second sensors located at approximately the same body site. In certain embodiments, first and second signals may be obtained by first and second sensors located at different body sites.

In certain embodiments, more than two signals are received at step 502. For example, PPG signals at three or more frequencies may be obtained at step 502. It will be noted that the steps of flowchart 500 may be applied to any number of received signals by application of the techniques described herein.

In certain embodiments, pre- or post-processing techniques are applied to one or more of the signals received at step 502. These techniques may include any one or more of the following: compressing, multiplexing, modulating, up-sampling, down-sampling, smoothing, taking a median or other statistic of the received signal, removing erroneous regions of the received signal, or any combination thereof.

In certain embodiments, the at least one signal received at step 502 are filtered using any suitable filtering technique. For example, a signal received at sensor 12 may be filtered by a low pass filter 68 prior to undergoing additional processing at microprocessor 48 within patient monitoring system 10. The low pass filter 68 may selectively remove frequencies that may later be ignored by a transformation or other processing step, which may advantageously reduce computational time and memory requirements. A signal received at step 502 may be high or band pass filtered to remove low frequencies. Such a filter may be, for example, a derivative filter. The signal received at step 502 may be filtered to remove a DC component. The signal received at step 502 may be normalized by dividing the signal by a DC component. The cutoff frequencies of a filter may be chosen based on the frequency response of the hardware platform underlying patient monitoring system 10.

Different operations, which may include transformation, processing and/or filtering techniques, may be applied to any one or more of the signals received at step 502 and/or any components of a multi-component signal. For example, different operations may be applied to a Red PPG signal and an IR PPG signal. An operation may be applied to a portion or portions of a received signal. An operation may be broken into one or more stages performed by one or more devices within signal processing system 700 (which may itself be a part of patient monitoring system 10). For example, a filtering technique may be applied by input signal generator 710 prior to passing the resulting input signal 716 to processor 712. Embodiments of the steps of flowchart 500 include any of the operations described herein performed in any suitable order.

In certain embodiments, signal 716 may be coupled to processor 712. Processor 712 may be any suitable software, firmware, and/or hardware, and/or combinations thereof for processing signal 716. For example, processor 712 may include one or more hardware processors (e.g., integrated circuits), one or more software modules, and computer-readable media such as memory, firmware, or any combination thereof. Processor 712 may, for example, be a computer or may be one or more chips (i.e., integrated circuits). Processor 712 may perform the calculations associated with the continuous wavelet transforms of the present disclosure as well as the calculations associated with any suitable interrogations of the transforms. Processor 712 may perform any suitable signal processing of signal 716 to filter signal 716, such as any suitable band-pass filtering, adaptive filtering, closed-loop filtering, and/or any other suitable filtering, and/or any combination thereof. Processor 716 may perform any suitable computation for signal analysis. For example, processor 716 may be capable of tuning a wavelet transform. In certain embodiments, processor 716 tunes the wavelet transform to a particular characteristic frequency to produce better definition in the wavelet transform for repeating signal features.

Processor 712 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 712 to, for example, store data corresponding to a continuous wavelet transform of input signal 716, such as data representing a scalogram. In one embodiment, data representing a scalogram may be stored in RAM or memory internal to processor 712 as any suitable three-dimensional data structure such as a three-dimensional array that represents the scalogram as energy levels in a time-scale plane. Any other suitable data structure may be used to store data representing a scalogram.

Processor 712 may be coupled to output 714. Output 714 may be any suitable output device such as, for example, one or more medical devices (e.g., a medical monitor that displays various physiological parameters, a medical alarm, or any other suitable medical device that either displays physiological parameters or uses the output of processor 712 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 700 may be incorporated into system 10 (FIGS. 1 and 2) in which, for example, input signal generator 710 may be implemented as parts of sensor 12 and monitor 14 and processor 712 may be implemented as part of monitor 14.

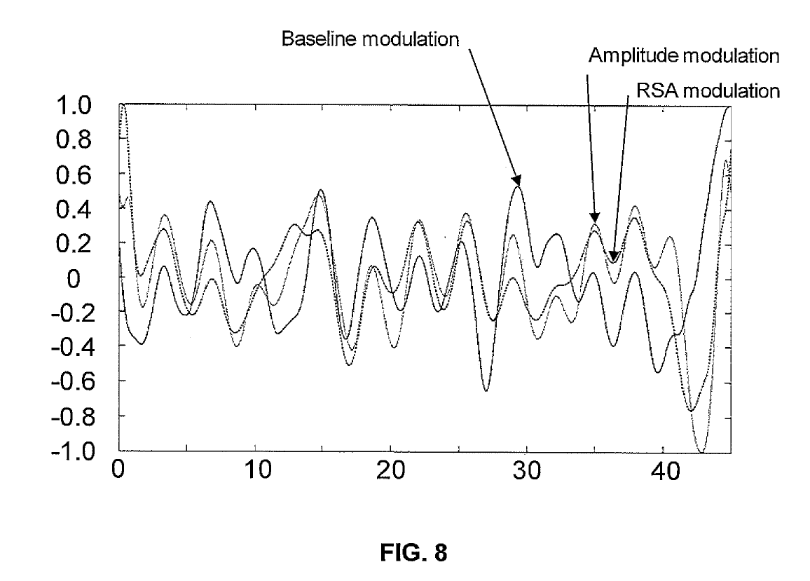

FIG. 8 is an illustrative plot showing the respiratory signal components from FIG. 3 aligned in phase. In this illustration, the baseline modulation signal is the reference signal, and the amplitude modulation signal and RSA modulation signal have been transformed, rotated, and inverted according to the method described in relation to FIG. 5. Upon close inspection, one can see that the amplitude modulation signal and RSA modulation signal are not merely time-shifted versions of the amplitude modulation signal and RSA modulation signal in FIG. 3. By rotating the signals in wavelet space, the waveforms themselves have changed so that signal noise is reduced.

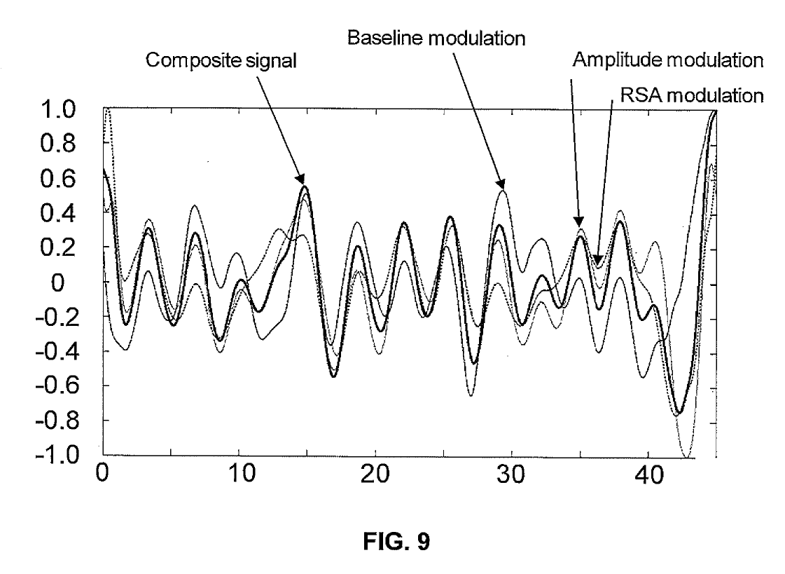

FIG. 9 is an illustrative plot showing a composite signal laid over the aligned respiratory signal components from FIG. 8. In this example, the three component waveforms were normalized and added together to form a composite respiration waveform. A respiration rate can be determined from the composite respiration waveform by analyzing the composite waveform to detect a frequency or periodicity indicative of respiration. As described above in relation to FIG. 5, any suitable method for deriving a composite signal or parameter may be used. This composite respiration signal may be displayed as a respiration signal on the monitor 14 with or without the component signals.

Some signals will have multiple components that have not been decoupled from each other. In these cases, rather than decoupling the components from each other so that individual signal components are transformed into the complex transform space, a multi-component signal can be transformed to a complex transform space. In the transformed space, rather than all points of the transformed signal being rotated, only points belonging to certain components of the signal may be rotated. Furthermore, not all points being rotated are necessarily rotated by the same phase shift value; rather, different phase shift values can be used for different components.

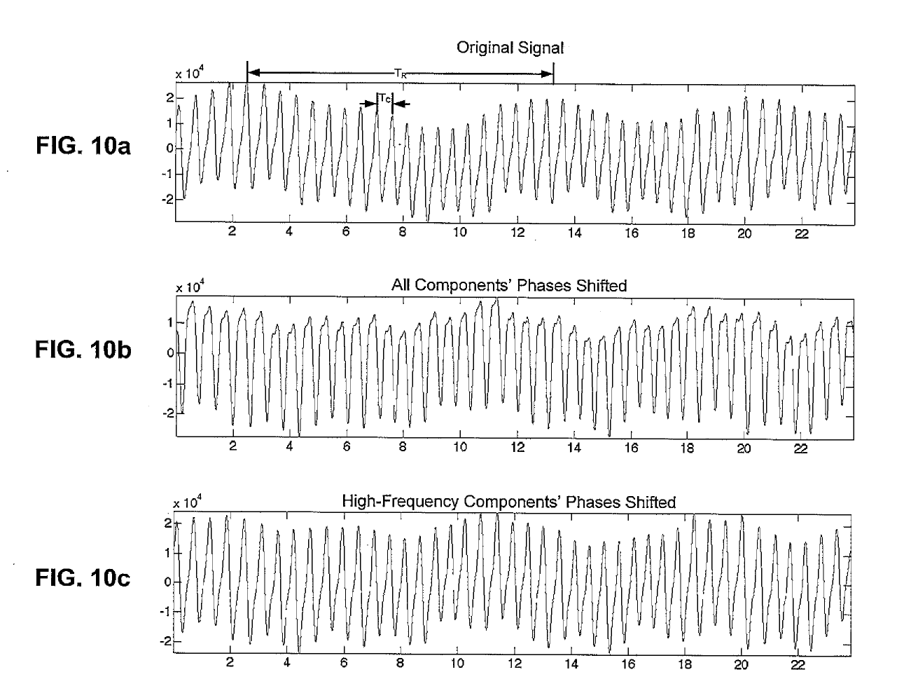

FIGS. 10a-10c illustrate aspects of rotating an entire transformed signal and rotating only a component of the transformed signal. FIG. 10a is a plot of a PPG signal received from a pulse oximeter. The PPG signal includes a high-frequency cardiac component, which appears as short pulses having periodicity T. The PPG signal also has a low-frequency respiratory component, which appears as the slower waves having periodicity T_{R}. FIG. 10b is an illustrative plot showing all points of the signal in FIG. 10a having undergone a phase shift equal to a quarter of the cycle of T_{R}. The peaks of the respiratory wave in FIG. 10b have been shifted relative to the peaks of the respiratory wave in FIG. 10a. However, shifting the entire signal has also affected the morphology of the cardiac pulses.

FIG. 10e is a plot showing the signal from FIG. 10a after just the low-frequency components have undergone a phase shift of one quarter of the cycle of T_{R}. In this case, the high-frequency components have not been altered. The entire signal from FIG. 10a was transformed into the complex transform space using a continuous wavelet transform. Then, only the respiratory components having a characteristic frequency below a given threshold (0.4 Hz in this case) were rotated. The entire transform signal, part of which was also rotated, was then inverted into the time domain to create the plot shown in FIG. 10c. The peaks of the respiratory wave in FIG. 10c have shifted with respect to peaks of the respiratory wave in FIG. 10a. However, unlike in the plot of FIG. 10b, the cardiac pulses have retained their original morphology from FIG. 10a.

It will be understood by those of skill in the art that aligning physiological signals may involve methods other than those described above. For example, other steps may be performed. Additionally, the systems and methods described herein may be used with other transformation and other realization of signals at multiple scales where an intrinsic period or periods may be determined.

It is to be understood that while systems, methods, and components have been described in conjunction with the various illustrative examples, the forgoing description is merely illustrative and does not limit the scope of the disclosure. While several examples have been provided in the present disclosure, it should be understood that the disclosed systems, components, and methods may be embodied in many other specific forms without departing from the scope of the present disclosure.

Variations and modifications will occur to those of skill in the art after reviewing this disclosure. The disclosed features may be implemented, in any combination and subcombinations (including multiple dependent combinations and sub-combinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

What is claimed is:

1. A method for defining a physiological parameter, comprising:
generating a transformed signal in a complex transform space, the transformed signal based on a first physiological signal, and the transformed signal having a magnitude and a phase;
generating a rotated signal by altering the phase of the transformed signal;
generating an inverted signal by inverting the rotated signal, wherein the inverted signal is aligned in phase with a second physiological signal; and
generating a combined signal indicative of the physiological parameter by combining the aligned inverted signal with the second physiological signal.

2. The method of claim 1, wherein at least one of the first physiological signal and the second physiological signal is a photoplethysmograph signal measured by a pulse oximeter.

3. The method of claim 2, wherein the photoplethysmograph signal includes at least one respiratory component.

4. The method of claim 3, wherein generating the transformed signal is done by transforming the photoplethysmograph signal using a continuous wavelet transform.

5. The method of claim 3, wherein generating the transformed signal is done by transforming the photoplethysmograph signal using a short time Fourier transform.

6. The method of claim 3, wherein a phase shift value is used to alter the phase of the transformed signal.

7. The method of claim 6, wherein the phase shift value is a minimum value of a median of distances between corresponding peaks of the first and second physiological signals.

8. The method of claim 3, further comprising aligning at least one additional physiological signal to the second physiological signal by:
generating an additional transformed signal in a complex transform space, the additional transformed signal based on the additional physiological signal, and the additional transformed signal having a magnitude and a phase;
generating an additional rotated signal by altering the phase of the additional transformed signal; and
generating an additional inverted signal by inverting the additional rotated signal, wherein the additional inverted signal is aligned in phase with the second physiological signal.

9. The method of claim 8, wherein the phase of the transformed signal is altered by a phase shift value that is different from a phase shift value for altering the additional transformed signal.

10. The method of claim 3, wherein transforming the first physiological signal into the complex transform space reduces noise in the signal.

11. The method of claim 3, wherein a first photoplethysmograph signal is received from a first sensing device positioned at a first part of a subject's body, and a second photoplethysmograph signal is received from a second sensing device positioned at a second part of the subject's body.

12. The method of claim 3, wherein combining the aligned inverted signal and the second physiological signal comprises normalizing and summing the signals.

13. The method of claim 3, further comprising extracting a rate of respiration from the combined signal.

14. The method of claim 3, wherein the transformed signal has a plurality of phases, and generating a rotated signal comprises altering more than one of the phases of the transformed signal.

15. A system for displaying a physiological parameter comprising:
a signal input configured to receive at least one physiological signal of a subject from a sensing device;
electronic processing equipment coupled to the signal input, the electronic processing equipment configured for:
receiving the at least one physiological signal;
aligning the at least one physiological signal with a reference physiological signal; and
generating a combined signal indicative of the physiological parameter by combining the at least one physiological signal with the reference physiological signal; and
a monitor for displaying a physiological parameter representative of the combined signal.

16. The system of claim 15, wherein aligning a first physiological signal with the reference physiological signal comprises:
generating a transformed signal in a complex transform space, the transformed signal based on the first physiological signal, and the transformed signal having a magnitude and a phase;
generating a rotated signal by altering the phase of the transformed signal; and
generating an inverted signal by inverting the rotated signal, wherein the inverted signal is aligned in phase with the reference physiological signal.

17. The system of claim 16, wherein the at least one physiological signal is a photoplethysmograph signal measured by a pulse oximeter.

18. The system of claim 17, wherein the photoplethysmograph signal includes at least one respiratory component.

19. The system of claim 16, wherein generating the transformed signal involves using a continuous wavelet transform.

20. The system of claim 16, wherein generating the transformed signal involves using a short time Fourier transform.

21. The system of claim 16, wherein a phase shift value is used to alter the phase of the transformed signal.

22. The system of claim 21, wherein the phase shift value is a minimum value of a median of the distances between corresponding peaks of the physiological signals.

23. The system of claim 16, wherein the electronic processing equipment is further configured for aligning at least one additional physiological signal to the reference physiological signal:

24. The system of claim 16, wherein the monitor is further configured to display the combined signal.

25. The system of claim 17, wherein:
the signal input is configured to receive a photoplethysmograph signal of the subject from a first sensing device and a photoplethysmograph signal of the subject from a second sensing device positioned at a different part of the body of the subject from the first device;
the first physiological signal is the signal input from the first sensing device; and
the reference physiological signal is the signal input from the second sensing device.

26. The system of claim 18, wherein the physiological parameter is a respiration rate.

27. The system of claim 16, wherein the transformed signal has a plurality of phases, and generating a rotated signal comprises altering more than one of the phases of the transformed signal.

28. Computer-readable medium for use in defining a physiological parameter, the computer-readable medium having computer program instructions recorded thereon for:
generating a transformed signal in a complex transform space, the transformed signal based on a first physiological signal, and the transformed signal having a magnitude and a phase;
generating a rotated signal by altering the phase of the transformed signal;
generating an inverted signal by inverting the rotated signal, wherein the inverted signal is aligned in phase with a second physiological signal; and
generating a combined signal indicative of the physiological parameter by combining the aligned inverted signal with the second physiological signal.
